(19) United States
(12) Patent Application Publication
(10) Pub. No.: US 2013/0190545 A1
(43) Pub. Date: Jul. 25, 2013

(54) METHOD AND PLANT FOR DEHYDRATION BY A DELIQUESCENT SUBSTANCE

(71) Applicants: Frédéric Augier, Saint Symphorien d'Ozon (FR); Maxime Vassieu, Cavaillon (FR)

(72) Inventors: Frédéric Augier, Saint Symphorien d'Ozon (FR); Maxime Vassieu, Cavaillon (FR)

(21) Appl. No.: 13/810,713

(22) PCT Filed: Jul. 21, 2011
(86) PCT No.: PCT/FR2011/000434

§ 371 Date: Mar. 12, 2013

(30) Foreign Application Priority Data
Jul. 23, 2010 (FR) 1003091

(51) Int. Cl. C07C 7/12

(52) U.S. Cl. CPC C07C 7/12
USPC 585/310; 208/88; 422/630

(57) ABSTRACT

The present invention relates to a method and to a plant for dehydration of a liquid or gaseous effluent, wherein the following stages are carried out:
neutralizing the effluent with at least one of the following brines: soda brine, potash brine, or mixtures thereof,
dehydrating the neutralized effluent on soda or potash briquettes,
collecting the brine resulting from dehydration and using it in the neutralization stage (2).

### FIELD OF THE INVENTION

The present invention relates to a method and to a plant for dehydration or drying of liquids by deliquescent dehydrating agents in form of briquettes, notably soda or potash briquettes, also referred to as alkaline solids. In particular, the object of the invention is to use in a process the brine resulting from drying or dehydration, said brine being referred to as alkaline solution.

### BACKGROUND OF THE INVENTION

The document “Liquid Drying by Solid Desiccant Materials: Experimental Study and Design Method”, F. Augier, C. Boyer and M. Vassieu, Oil & Gas Science and Technology—Rev. IFP, Vol.63 (2008), No.6, pp. 713-722, describes drying with deliquescent substances.

Using salts or brine for dehydration is well known, but the method and the device according to the present invention have not been described.

### SUMMARY OF THE INVENTION

The present invention thus relates to a method for dehydration of a liquid or gaseous effluent wherein the following stages are carried out:
neutralizing said effluent with at least one of the following brines: soda brine, potash brine, or mixtures thereof,
dehydrating said neutralized effluent on soda or potash briquettes,
collecting the brine resulting from dehydration and using it in said neutralization stage.

According to the method, the dimensions of the briquettes can range between 1 and 3 cm.

In the method, the effluent can be a hydrocarbon, such as LPG, a gasoline, a diesel oil, a kerosene.

The invention also relates to a plant for dehydration of a liquid or gaseous effluent, comprising:
effluent neutralization means using at least one of the following brines: soda brine, potash brine, or mixtures thereof,
neutralized effluent dehydration means comprising soda or potash briquettes,
means for collecting the brine resulting from dehydration and means for recycling the brine to the neutralization means.

### BRIEF DESCRIPTION OF THE FIGURES

Other features and advantages of the invention will be clear from reading the description hereafter of embodiments given by way of non-limitative example, with reference to the accompanying figures wherein:

FIG. 1 diagrammatically shows the principle of the invention, and

FIG. 2 illustrates the invention applied to hydrocarbon sweetening methods using sulfur compounds removal.

### DETAILED DESCRIPTION

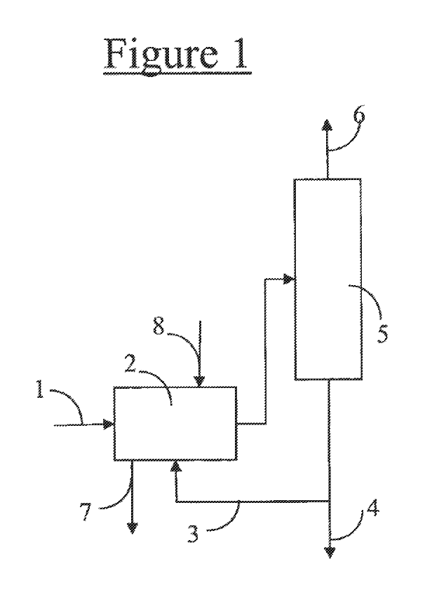

FIG. 1 shows the general principle of a method wherein a product to be neutralized and dried is fed through a line 1 into neutralization means 2 where an alkaline solution is used for neutralization or sweetening of compounds present in the product. The product thus treated enters a drying column 5 filled with alkaline solids, or deliquescent substances, of same chemical nature as the alkaline solution used in means 2. Upon contact with the alkaline solids, the product to be treated dehydrates by forming an alkaline solution that flows to the bottom of drying column 5 via line 4. The treated product leaves column 5 through an upper line 6. Part or all of the alkaline solution leaving the bottom of column 5 is recycled to sweetening means 2 via line 3. This recycling does not preclude adding make-up alkaline solution into drum 2 via a line 8. An alkaline solution purge 7 is also possible. The advantage of using in the drying column a solid of same nature as the alkaline solution used in the neutralization process is that it allows to limit the fresh alkaline solution supply while decreasing the amount of effluent flowing from the bottom of the drying column.

Preferably, products marketed by the Newton's Company (France) in form of soda (NaOH) or potash (KOH) grains are used. Their specific briquette shapes enable optimum grain behaviour as the product is being consumed.

The briquette shape affords a larger specific surface area than a bead of equal weight or volume, while limiting surface losses due to contact between grains as it is the case with flaked shapes. It is an optimum shape for the specific surface area really available.

The briquette shape allows rearrangement of the bed as it is being consumed. The bed height progressively decreases until the minimum bed height required by the dehydration method is reached.

The deliquescent salts absorb the moisture and dissolve in the absorbed water so as to form a brine of a mixture of salt and water.

The brines generated by the dehydration thus are soda or potash brines at concentrations ranging between 20 and 60 mass %.

In particular mercaptan extraction methods using a soda solution are particularly pertinent within the context of the invention because they allow great benefits to be gained therefrom. These methods are commonly used in refineries in order to extract some sulfur compounds, mercaptans for example, from various hydrocarbons, such as LPG, gasoline, diesel oil, kerosene. There are many variants of this type of method, depending on the type of product to be treated and on the quality of the desired end product.

These methods generally comprise the following operations:
a prewash stage using diluted soda (between 1 and 10%), intended for neutralization of the acids, H₂S for example, at the process inlet,
countercurrent extraction using diluted soda (between 10 and 20%), comprising a small amount of catalyst (not active in this stage), the mercaptans (RSH) are then dissociated into mercaptides (RS-),
a stage of oxidation in air of the mercaptides to disulfides, wherein the catalyst present in the soda is active,
a stage of extraction of the disulfides by a hydrocarbon, followed by decantation in 1 or 2 stages,
recycling of the regenerated soda to the top of the liquid-liquid extraction column.

Once treated, the feedstock is generally washed with water. The washed feedstock is then passed through a sand filter in order to separate it from the free water.

The residual water content of the feedstock ranges, according to filters from the prior art, between 50 and 100 ppm.

According to the invention, the sand filter is replaced by a drying drum containing a deliquescent solid consisting of solid soda pellets. This affords several advantages:
the dehydration performed is then deeper because it is not limited to the free water. A final water content ranging between 10 and 50 ppm can be obtained depending on the nature of the feedstocks,
the soda pellets used generate a brine that is recovered at the bottom of the drying column. This brine can be diluted and used either in the soda prewash column or as makeup in the extraction loop. This is an important advantage in relation to the use of sand filters because, in the latter case, the free water extracted does not come in form of brine and it has to be treated. When using soda pellets, the concentrated brine collected at the bottom of the drying column can optionally be re-used outside the process if there is a stream to be neutralized for example on the industrial site, but recycling it within the process is much more interesting. Indeed, recycling the brine to the process wherein it is produced allows:

1) to produce brine only when the process is operating, so as to avoid using storage tanks inherent in external upgrading,

2) the brine to be produced in the geographical vicinity of its place of use, hence pipe and pump savings.

Passage of the feedstock through a highly (pure) soda-concentrated bed can allow to determine the traces of H₂S or even of mercaptans still present therein.

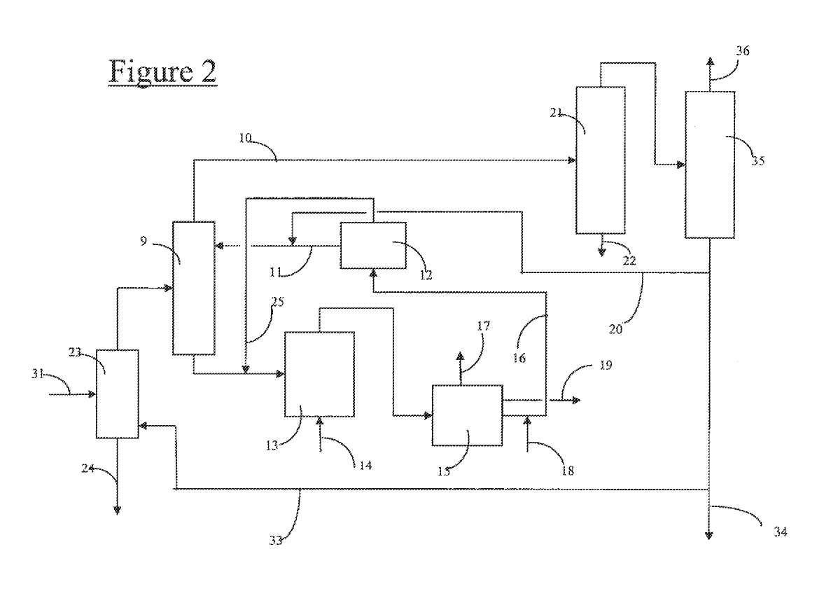

FIG. 2 shows an example of a method of extracting mercaptans present in a hydrocarbon implementing the invention. The feedstock or product to be treated, which contains H₂S and mercaptans among other things, flows through line 31 into a prewash column 23 containing diluted soda. This column 23 is used to remove the major part of the H₂S present in the feedstock, and it also removes at least a small part of the mercaptans. The effluent flowing out through line 24 contains diluted soda, H₂S, mercaptans and other acids initially present in the feedstock. The product flowing from the top of column 23 then enters a liquid-liquid extraction column 9. Extraction column 9 is also supplied with a soda solution via line 11, diluted between 10 and 20 mass %. Extraction is generally carried out under countercurrent conditions, where the hydrocarbon feedstock flows upwards and the soda solution flows downwards. The great majority of the mercaptans is then extracted in the soda phase, in form of mercaptides. The mercaptide-laden soda solution is then sent to an oxidation reactor 13 for converting the mercaptides to disulfides. Oxidation is carried out by means of an air injection 14. Oxidation is achieved through homogeneous-phase catalysis. The catalyst used preferably belongs to the phthalocyanine family, and it is dissolved in the soda phase. The multiphase mixture leaving reactor 13 enters a separation drum 15. Drum 15 is used for separating the gas phase, which leaves through line 17, the soda phase via line 16 and an organic phase via line 19 containing the disulfides. The method is based on the highly hydrophobic character of the disulfides that are very readily extracted by a hydrocarbon phase. A hydrocarbon, a gasoline for example, is therefore injected into soda circuit 16 via line 18. This hydrocarbon extracts the disulfides present in the soda. The soda phase and the gasoline phase are separated in a settling drum 12. The soda thus regenerated is sent back to extraction column 9 via line 11. The gasoline leaves settling drum 12 through line 25 and it is mixed with the soda leaving extraction drum 9. The gasoline then flows through oxidation reactor 13 and extracts the disulfides that have formed therein. Fresh gasoline injection at the outlet of drum 15 allows to complete the disulfide extraction and thus to limit sulfur backflow in the extraction column.

The desulfurized product leaving the extraction column is sent through line 10 to a water wash column 21. An aqueous effluent leaves bottom 22 of this column. The washed product is then fed into a drying column 35. Within the context of the invention, this column is filled with solid soda. The dehydrated product leaves column 35 via line 36. The drying column produces a soda solution that leaves column 35 via line 34 and whose concentration is higher than that in prewash column 23 or in extraction column 9. A fraction or all of the soda solution produced upon dehydration can be recycled to the process: either to the prewash column via line 33 or to any point of the soda solution loop passing through extraction column 9, oxidation reactor 13 and separation drums 15 or 12. In FIG. 2, part of the soda solution produced in drying column 35 is recycled via line 20 to line 11 just at the inlet of extraction column 9. The soda solution can be diluted with water prior to injection so as to be used with the most pertinent concentration.

A pertinent implementation of the invention consists in recycling the brine to prewash column 23 only when the hydrocarbon feedstock is highly concentrated in mercaptans. It is not uncommon for the mercaptan concentration in the hydrocarbon to be treated to fluctuate, therefore starting recycling the brine when the feedstock to be treated is concentrated allows to stabilize the overall operation of the process and thus to improve the performances thereof.

Another interesting implementation consists in permanently recycling the brine to prewash column 23. Thus, in comparison with the operation without recycling, the brine consumption is identical but the performances of the process are improved as a result of a prewash operating on average at a higher soda concentration. The sulfur-containing species such as mercaptans, COS or H₂5 are thus more efficiently captured by the aqueous phase during prewash.

The advantages of the invention can thus be either to save soda or to improve the overall performances of the global desulfurization method.

1. A method for dehydration of a liquid or gaseous effluent wherein the following stages are carried out:
neutralizing said effluent with at least one of the following brines: soda brine, potash brine, or mixtures thereof,
dehydrating said neutralized effluent on soda or potash briquettes,
collecting the brine resulting from dehydration and using it in said neutralization stage.

2. A method as claimed in claim 1, wherein the dimensions of said briquettes range between 1 and 3 cm.

3. A method as claimed in any one of claim 1, wherein said effluent is a hydrocarbon, such as LPG, a gasoline, a diesel oil, a kerosene.

4. A plant for dehydration of a liquid or gaseous effluent, comprising:
means for neutralizing said effluent using at least one of the following brines: soda brine, potash brine, or mixtures thereof,
means for dehydrating said neutralized effluent comprising soda or potash briquettes,
means for collecting the brine resulting from dehydration and means for recycling said brine to the neutralization means.
